SEQUENCE LISTING (10 sequences)

SEQ ID NO: 1 - DNA, 20 bp, synthetic construct
  source 1..20: mol_type = other DNA
ggccttccctacttcacaag

SEQ ID NO: 2 - DNA, 20 bp, synthetic construct
  source 1..20: mol_type = other DNA
atttccacgatttcccagag

SEQ ID NO: 3 - DNA, 22 bp, synthetic construct
  source 1..22: mol_type = other DNA
ctggtgtgtgacgttcccatta

SEQ ID NO: 4 - DNA, 18 bp, synthetic construct
  source 1..18: mol_type = other DNA
ccgacagcacgaggcttt

SEQ ID NO: 5 - DNA, 22 bp, synthetic construct
  source 1..22: mol_type = other DNA
gaccctcacactcagatcatct

SEQ ID NO: 6 - DNA, 20 bp, synthetic construct
  source 1..20: mol_type = other DNA
cctccacttggtggtttgct

SEQ ID NO: 7 - DNA, 19 bp, synthetic construct
  source 1..19: mol_type = other DNA
ccagcctactcattgggat

SEQ ID NO: 8 - DNA, 19 bp, synthetic construct
  source 1..19: mol_type = other DNA
gggcctgctgttcacagtt

SEQ ID NO: 9 - DNA, 20 bp, synthetic construct
  source 1..20: mol_type = other DNA
acccagaagactgtggatgg

SEQ ID NO: 10 - DNA, 20 bp, synthetic construct
  source 1..20: mol_type = other DNA
acacattgggggtaggaaca

(19) United States
(12) Patent Application Publication
(10) Pub. No.: US 2025/0288627 A1
(43) Pub. Date: Sep. 18, 2025

(54) COMPOSITION FOR AMELIORATING, PREVENTING OR TREATING SYSTEMIC SCLEROSIS AND PULMONARY FIBROSIS CONTAINING BIFIDOBACTERIUM LONGUM RAPO

(71) Applicants: INDUSTRY-ACADEMIC COOPERATION FOUNDATION GYEONGSANG NATIONAL UNIVERSITY, Jinju-si (KR); BIFIDO CO., LTD., Hongcheon-gun (KR)

(72) Inventors: Sang Il LEE, Jeonju-si (KR); Myeong Soo PARK, Seoul (KR)

(73) Assignees: INDUSTRY-ACADEMIC COOPERATION FOUNDATION GYEONGSANG NATIONAL UNIVERSITY, Jinju-si (KR); BIFIDO CO., LTD., Hongcheon-gun (KR)

(21) Appl. No.: 18/975,096

(22) Filed: Dec. 10, 2024

(30) Foreign Application Priority Data
Mar. 13, 2024 (KR) 10-2024-0035381

(51) Int. Cl. A61K 35/745; A61K 35/00; A61P 11/00

(52) U.S. Cl. CPC A61K 35/745; A61P 11/00; A61K 2035/115

(57) ABSTRACT

Disclosed is a method for ameliorating, preventing or treating systemic sclerosis and pulmonary fibrosis, including administering to a subject in need thereof a composition containing Bifidobacterium longum RAPO (KCTC13773BP) as an active ingredient. The composition controls infiltration and inflammatory response of inflammatory monocytes and macrophages, and facilitates an increase in short-chain fatty acid producing microorganisms, thus being effective in ameliorating, preventing or treating systemic sclerosis and pulmonary fibrosis.

### CROSS-REFERENCE TO RELATED APPLICATIONS

This application claims priority to Korean Patent Application No. 10-2024-0035381 filed Mar. 13, 2024, the entire disclosure of which is incorporated herein by reference.

### INCORPORATION BY REFERENCE OF SEQUENCE LISTING

The content of the electronically submitted sequence listing, file name: Q304622_sequence listing as filed; size: 9,843 bytes; and date of creation: Dec. 10, 2024, filed herewith, is incorporated herein by reference in its entirety.

### BACKGROUND OF THE INVENTION

### Field of the Invention

The present invention relates to a method for ameliorating, preventing or treating systemic sclerosis and pulmonary fibrosis, comprising administering to a subject in need thereof a composition comprising Bifidobacterium longum RAPO (KCTC13773BP), and more particularly to a composition having the efficacy of ameliorating, preventing or treating systemic sclerosis and pulmonary fibrosis based on the anti-inflammatory anti-fibrotic effects and mechanisms of Bifidobacterium longum RAPO.

### Description of the Related Art

Systemic sclerosis (SSc) is an autoimmune disease characterized by severe fibrosis of the skin and internal organs and vascular occlusion due to functional/structural abnormalities in small blood vessels and is an intractable rare disease that causes serious complications such as pulmonary fibrosis and pulmonary hypertension. In particular, interstitial lung disease (ILD) is a typical complication that occurs in 55 to 80% of systemic sclerosis patients and is known to be the greatest cause of mortality in the systemic sclerosis patients.

Interstitial lung disease progresses to pulmonary fibrosis, which causes severe respiratory distress due to excessive proliferation of fibroblasts in the process of lung tissue damage and inflammation. The most common pulmonary fibrosis is similar to idiopathic pulmonary fibrosis (IPF), the cause of which has not been clearly identified to date and which is an intractable disease in which lung tissue cannot be restored once it has been damaged.

The exact cause of systemic sclerosis is not yet known, but inflammation, immune abnormalities, and vasculopathy are considered major contributing factors, and recent research has suggested that the microbiome is one of the pathogenic factors of systemic sclerosis. Previous research shows that a decrease in microorganisms producing butyrate, which is a type of short-chain fatty acid, is found in the intestines of systemic sclerosis patients, and that skin and pulmonary fibrosis are suppressed in mice with systemic sclerosis and pulmonary fibrosis after administration of sodium butyrate, which is known as a microbial fermentation metabolite.

As there has been no drug effective for systemic sclerosis and pulmonary fibrosis to date, there is a high demand for the development of novel drugs. Therefore, there is a need for development of novel drugs that are capable of effectively treating systemic sclerosis and pulmonary fibrosis while being safe for the human body.

### PRIOR ART LITERATURE

### Patent Literature

(Patent literature 1) Korean Patent Laid-open Publication No. 10-2023-0031304 (publication date: Mar. 7, 2023) relates to a composition for treating pulmonary fibrosis containing a compound having excellent therapeutic efficacy for idiopathic pulmonary fibrosis (IPF) of unknown cause or pulmonary fibrosis caused by coronavirus infection-19.

### SUMMARY OF THE INVENTION

Therefore, the present invention has been made in view of the above problems, and it is an object of the present invention to provide a composition for ameliorating, preventing or treating systemic sclerosis and pulmonary fibrosis containing Bifidobacterium longum RAPO (KCTC13773BP) as an active ingredient.

In accordance with an aspect of the present invention, the above and other objects can be accomplished by the provision of a food composition for ameliorating systemic sclerosis containing Bifidobacterium longum RAPO (KCTC13773BP) as an active ingredient.

Meanwhile, the food composition may be used for ameliorating systemic sclerosis and pulmonary fibrosis.

In accordance with another aspect of the present invention, there is provided a pharmaceutical composition for preventing or treating systemic sclerosis containing Bifidobacterium longum RAPO (KCTC13773BP) as an active ingredient.

Meanwhile, the pharmaceutical composition may be used for preventing or treating systemic sclerosis and pulmonary fibrosis.

In accordance with another aspect of the present invention, there is provided an animal food composition for ameliorating systemic sclerosis containing Bifidobacterium longum RAPO (KCTC13773BP) as an active ingredient.

Meanwhile, the animal food composition may be used for preventing or treating systemic sclerosis and pulmonary fibrosis.

In accordance with another aspect of the present invention, there is provided an animal drug for ameliorating systemic sclerosis containing Bifidobacterium longum RAPO (KCTC13773BP) as an active ingredient.

Meanwhile, the animal drug may be used for preventing or treating systemic sclerosis and pulmonary fibrosis.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a graph showing the result of evaluation of the antifibrotic efficacy of Bifidobacterium strains in mice with systemic sclerosis (*P<0.05 vs PBS);

FIG. 2 is a graph showing the result of evaluation of the therapeutic efficacy for skin and associated pulmonary fibrosis in mice with systemic sclerosis of Bifidobacterium longum RAPO (KCTC13773BP) (A to C) and the result of evaluation of the therapeutic efficacy for pulmonary fibrosis in mice with idiopathic pulmonary fibrosis of Bifidobacterium longum RAPO (KCTC13773BP) (D to E) (*P<0.05, **P<0.01);

FIG. 3 is a graph showing the result of evaluation of the therapeutic efficacy for pulmonary fibrosis in mice with idiopathic pulmonary fibrosis of single/combination administration of a conventional drug for pulmonary fibrosis and Bifidobacterium longum RAPO (KCTC13773BP) (*P<0.05);

FIG. 4 is a graph showing the result of evaluation of inhibition of expression of fibrosis marker proteins in mice with systemic sclerosis of Bifidobacterium longum RAPO (KCTC13773BP) (*P<0.05, **P<0.01);

FIG. 5 is a graph showing the results of analysis of macrophages in the skin (A) and analysis of morphological changes in macrophages in the bronchoalveolar fluid (B) in mice with systemic sclerosis upon administration of Bifidobacterium longum RAPO (KCTC13773BP) (*P<0.05, **P<0.01);

FIG. 6 is a graph showing the results of analysis of inflammatory substances in the bronchoalveolar fluid (A), evaluation of expression of fibrosis/inflammatory marker genes in the skin and lung tissue (B), and analysis of inflammatory mononuclear cells in the spleen (C) in mice with systemic sclerosis upon administration of Bifidobacterium longum RAPO (KCTC13773BP) (*P<0.05, **P<0.01);

FIG. 7 is a graph showing the results of evaluation of the intestinal microbial diversity index (A) and changes in the intestinal microbial population distribution (B) in mice with systemic sclerosis upon administration of Bifidobacterium longum RAPO (KCTC13773BP) (*P<0.05, **P<0.01);

FIG. 8 is a graph showing relative distribution of short chain fatty acids (SCFAs)-producing microorganisms in mice with systemic sclerosis upon administration of Bifidobacterium longum RAPO (KCTC13773BP) (*P<0.05, **P<0.01);

FIG. 9 is a graph showing the results of evaluation of expression of inflammatory marker genes in macrophages in mice with systemic sclerosis upon administration of Bifidobacterium longum RAPO (KCTC13773BP) (*P<0.05, **P<0.01); and

FIG. 10 is a graph showing the result of evaluation of expression of α-SMA protein in fibroblasts in mice with systemic sclerosis upon administration of Bifidobacterium longum RAPO (KCTC13773BP) (*P<0.05, **P<0.01).

### DETAILED DESCRIPTION OF THE INVENTION

Systemic sclerosis is an autoimmune disease characterized by severe fibrosis of the skin and internal organs and vascular occlusion due to functional/structural abnormalities in small blood vessels and is an intractable rare disease that causes serious complications such as pulmonary fibrosis and pulmonary hypertension. In particular, interstitial lung disease is known to be the greatest cause of mortality in the systemic sclerosis patients. Interstitial lung disease progresses to pulmonary fibrosis, which causes severe respiratory distress due to excessive proliferation of fibroblasts in the process of lung tissue damage and inflammation. The most common pulmonary fibrosis is similar to idiopathic pulmonary fibrosis (IPF), the cause of which has not been clearly identified to date and which is an intractable disease in which lung tissue cannot be restored once it has been damaged. However, as there has been no drug effective for systemic sclerosis and pulmonary fibrosis to date, there is a high demand for the development of novel drugs. The present invention provides a new drug developed for treating systemic sclerosis and pulmonary fibrosis.

Previous research shows that a decrease in butyrate-producing microorganisms is found in the intestines of systemic sclerosis patients, and that skin and pulmonary fibrosis is suppressed in mice with systemic sclerosis and pulmonary fibrosis after administration of sodium butyrate, known as a microbial fermentation metabolite. Accordingly, in the present invention, the antifibrotic efficacy of Bifidobacterium species, which have the ability to produce short-chain fatty acids (SCFAs) containing butyrate and have been proven to be safe, was evaluated in mice with systemic sclerosis and pulmonary fibrosis. Whether or not various Bifidobacterium strains effectively alleviate systemic sclerosis and pulmonary fibrosis was tested. Among them, Bifidobacterium longum RAPO (KCTC13773BP), which had the best efficacy, was used to provide a composition that exhibits excellent efficacy in ameliorating, preventing, or treating systemic sclerosis and pulmonary fibrosis.

Accordingly, the present invention provides a food composition for ameliorating systemic sclerosis containing Bifidobacterium longum RAPO (KCTC13773BP) as an active ingredient. Meanwhile, the food composition is preferably used for ameliorating systemic sclerosis and pulmonary fibrosis.

In addition, the present invention provides a pharmaceutical composition for preventing or treating systemic sclerosis containing Bifidobacterium longum RAPO (KCTC13773BP) as an active ingredient. Meanwhile, in the pharmaceutical composition of the present invention, the pharmaceutical composition is preferably used for preventing or treating systemic sclerosis and pulmonary fibrosis.

In addition, the present invention provides an animal food composition for ameliorating systemic sclerosis containing Bifidobacterium longum RAPO (KCTC13773BP) as an active ingredient. Meanwhile, in the animal food composition of the present invention, the animal food composition is preferably used for ameliorating systemic sclerosis and pulmonary fibrosis.

In addition, the present invention provides an animal drug for preventing or treating systemic sclerosis containing Bifidobacterium longum RAPO (KCTC13773BP) as an active ingredient. Meanwhile, in the animal drug of the present invention, the animal drug is preferably used for preventing or treating systemic sclerosis and pulmonary fibrosis. The Bifidobacterium longum RAPO used herein is a strain deposited with the Korea Research Institute of Bioscience and Biotechnology on Dec. 11, 2018 and under the accession number KCTC13773BP, which is a strain disclosed in Korea Patent No. 10-2074445 (registration date: Jan. 31, 2020).

Meanwhile, in the present invention, several experiments were conducted in animal models to determine whether or not the composition can ameliorate, prevent, or treat systemic sclerosis and pulmonary fibrosis. The result shows that Bifidobacterium longum RAPO of the present invention is much more effective in ameliorating skin and pulmonary fibrosis in mice with systemic sclerosis than other Bifidobacterium strains. The results of re-experiments are identical and show that Bifidobacterium longum RAPO also has an efficacy in alleviating pulmonary fibrosis in mice with idiopathic pulmonary fibrosis.

In addition, it was found that administration of a combination of pirfenidone (PFD), which is a conventional drug for pulmonary fibrosis, and Bifidobacterium longum RAPO exhibits a synergistic effect in treating pulmonary fibrosis, compared to administration of PFD alone, and it was found that the administration of combination significantly reduces the number of inflammatory monocytes or macrophages infiltrated in the spleen, skin, and bronchoalveolar cavities, while suppressing overexpression of inflammatory and fibrotic genes in the skin and lungs. In addition, it was found that Bifidobacterium longum RAPO restores the diversity of intestinal microorganisms and increases the distribution of bacteria producing short-chain fatty acids beneficial for disease recovery, and that Bifidobacterium longum RAPO and a metabolite thereof suppress inflammatory responses in macrophages and inhibit differentiation of fibroblasts into myofibroblasts.

As such, the Bifidobacterium longum RAPO according to the present invention is capable of regulating infiltration and inflammatory responses of inflammatory monocytes and macrophages and of facilitating the increase of short-chain fatty acid-producing microorganisms, thus exhibiting excellent effects in ameliorating, preventing, or treating systemic sclerosis and pulmonary fibrosis and being a promising pharmabiotic target.

Meanwhile, the food composition or animal feed composition according to the present invention may be, for example, any one selected from meat, grains, caffeinated beverages, general drinks, chocolate, bread, snacks, confectionery, candy, pizza, jellies, noodles, gums, ice cream, alcoholic beverages, alcohol, vitamin complexes and other health supplements, but is not limited thereto.

In addition, the amount of Bifidobacterium longum RAPO (KCTC13773BP) strain in the food composition or animal food composition of the present invention is not limited and may be varied depending on the condition of the administration subject, the type of specific disease, the degree of progression, or the like. If necessary, the amount of Bifidobacterium longum RAPO (KCTC13773BP) strain may also be the same as the total content of the food. In the present invention, the Bifidobacterium longum RAPO (KCTC13773BP) strain is preferably orally administered in an amount of 1×10⁸ CFU/200 [symbol] to 2×10⁹ CFU/200 [symbol] to a mouse model target.

When the food composition or animal feed composition according to the present invention is used as a food additive, it may be added as it is or used in combination with other foods or food ingredients, and may be used appropriately in accordance with a conventional method.

Meanwhile, the pharmaceutical composition or animal drug according to the present invention may further contain a pharmaceutically acceptable carrier, diluent, or excipient. The pharmaceutically acceptable carrier, excipient, or diluent includes lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinylpyrrolidone, water, methylhydroxybenzoate, propylhydroxybenzoate, talc, magnesium stearate, and mineral oil. These may be used singly or in combination. In addition, the pharmaceutical composition may further contain a filler, an anti-agglomerant, a lubricant, a wetting agent, a fragrance, an emulsifier or a preservative when the therapeutic or prophylactic agent is a drug.

Meanwhile, the formation of the pharmaceutical composition or animal drug of the present invention may be prepared in a desired form depending on the method of use, and may be particularly prepared using a method known in the art selected to provide rapid, sustained, or delayed release of the active ingredient after administration to a mammal. For example, the formulation may be selected from plasters, granules, lotions, liniments, lemonades, aromatic waters, powders, syrups, ophthalmic ointments, liquids and solutions, aerosols, extracts, elixirs, ointments, fluidextracts, emulsions, suspensions, decoctions, infusions, ophthalmic solutions, tablets, suppositories, injections, spirits, cataplasms, capsules, creams, troches, tinctures, pastes, pills, and soft or hard gelatin capsules.

Meanwhile, the dosage of the pharmaceutical composition or animal drug of the present invention is preferably determined in consideration of the administration method, the age, gender, and weight of the patient, and the severity of the disease. However, the dosage effective for the desired treatment may be easily changed depending on the condition of the user and the prescription of doctors. In addition, the pharmaceutical composition or animal drug of the present invention is preferably administered once per day or in a portionwise manner per week or month.

In addition, the pharmaceutical composition or animal drug according to the present invention may be administered as an individual therapeutic agent or in combination with other therapeutic agents, and may be administered sequentially or simultaneously with conventional therapeutic agents. Taking all of the factors into consideration, the pharmaceutical composition may be administered in a minimum amount that can obtain the maximum effect without causing side effects, which can be easily determined by those skilled in the art.

Hereinafter, the present invention will be described in more detail with reference to the following examples and experimental examples. The scope of the present invention is not limited to the examples and experimental examples, and encompasses modifications of the technical concept equivalent thereto.

### Example 1: Analysis of Efficacy of Bifidobacterium longum RAPO (KCTC13773BP) in Inhibiting Skin and Pulmonary Fibrosis in Animal Test Model (Mouse)

### 1) Analysis of Efficacy of Bifidobacterium Strains in Systemic Sclerosis Test Model

Probiotic strains of the Bifidobacterium series, namely, Bifidobacterium bifidum BGN4 (KCCM12754P), Bifidobacterium longum BORI (KCCM10492), and Bifidobacterium longum RAPO (KCTC13773BP) were administered to mice with systemic sclerosis disease induced by bleomycin (BLM) and the efficacy thereof was analyzed.

For this purpose, 8-week-old male C57BL/6 mice were used to induce systemic sclerosis and 1 mg/mL of bleomycin was injected subcutaneously into the dorsal area at 100 μL per mouse, 5 times a week for a total of 2 weeks to induce skin and accompanying pulmonary fibrosis. Bifidobacterium strains (Bifidobacterium bifidum BGN4, Bifidobacterium longum BORI, and Bifidobacterium longum RAPO) were administered orally at 2×10⁹ CFU/200 μL to each mouse 5 times a week for a total of 4 weeks, starting from 2 weeks before inducing systemic sclerosis with BLM until the end of the experiment, which is 2 weeks after BLM administration. PBS containing no Bifidobacterium strain was orally administered as a control group. Efficacy evaluation was performed as follows. Skin and lung tissues of mice with fibrosis induced on the 14^{th} day after the start of BLM administration were sampled and fixed with formalin to form paraffin tissue blocks. The formed paraffin blocks were cut into 5 μm thick sections with a microtome and stained with Masson-Trichrome (MT), and the degree of fibrosis in the skin and lungs was evaluated. The degree of fibrosis was evaluated by measuring a dermal thickness for the skin and an Ashcroft score, a collagen area, and an alveoli area for the lungs.

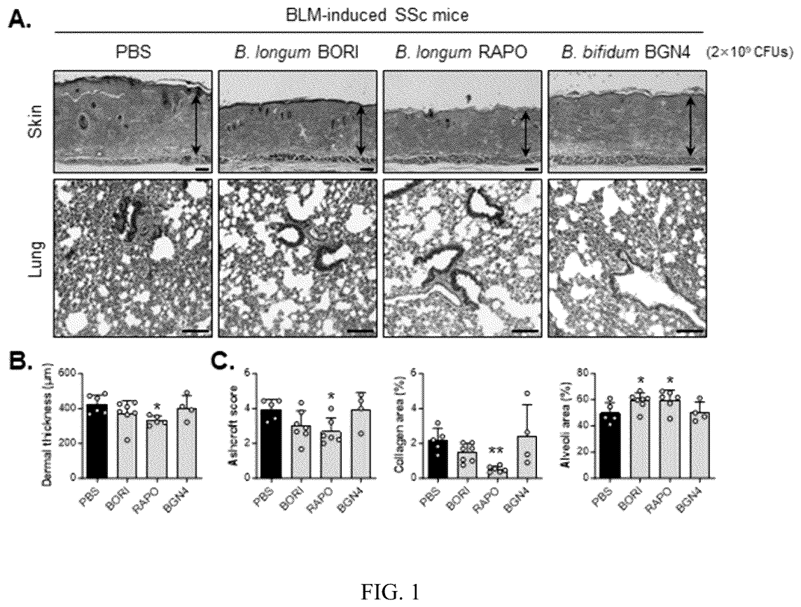

As shown in FIG. 1, the result shows that, compared to the group not administered the strain (PBS), the group administered Bifidobacterium longum BORI (BORI group) was slightly effective in alleviating pulmonary fibrosis, such as an increase in alveoli area, and the group administered Bifidobacterium longum RAPO (RAPO group) exhibited a decrease in skin thickness, Ashcroft score, and collagen area, and an increase in alveoli area, which indicates that Bifidobacterium longum was highly effective in improving both skin and pulmonary fibrosis. Meanwhile, the group administered Bifidobacterium bifidum BGN4 (BGN4 group) did not have any inhibitory effect on skin and pulmonary fibrosis.

2) Analysis of Efficacy of Bifidobacterium longum RAPO (KCTC13773BP) on Inhibition of Skin and Pulmonary Fibrosis in Systemic Sclerosis/Idiopathic Pulmonary Fibrosis Test Models

To determine whether or not the efficacy of Bifidobacterium longum RAPO, which had the best efficacy on skin and pulmonary fibrosis in systemic sclerosis disease mice as described above, was consistent, the efficacy was evaluated again in systemic sclerosis and idiopathic pulmonary fibrosis (IPF) mice.

The method of producing systemic sclerosis mice and administering and of evaluating the efficacy of Bifidobacterium longum RAPO was the same as in the experiment (FIG. 1). The method of producing mice with idiopathic pulmonary fibrosis and of evaluating the efficacy thereon was as follows: 50 μL (1 U/mouse kg) of BLM was injected subcutaneously once into a trachea to induce idiopathic pulmonary fibrosis in 8-week-old male C57BL/6 mice. Bifidobacterium longum (RAPO) was administered orally at 2×10⁹ CFU/200 μL to each mouse 5 times a week for a total of 4 weeks, starting from 7 days before inducing systemic sclerosis with BLM to the end of the experiment, which is the 21st day after BLM administration. PBS containing no Bifidobacterium longum (RAPO) was orally administered as a control group. Efficacy evaluation was performed as follows. Skin and lung tissues of mice with fibrosis induced on the 21st day after the start of BLM administration were sampled and fixed with formalin to form paraffin tissue blocks. The formed paraffin blocks were cut into 5 μm thick sections with a microtome and stained with Masson-Trichrome (MT), and the degree of fibrosis in the skin and lungs was evaluated.

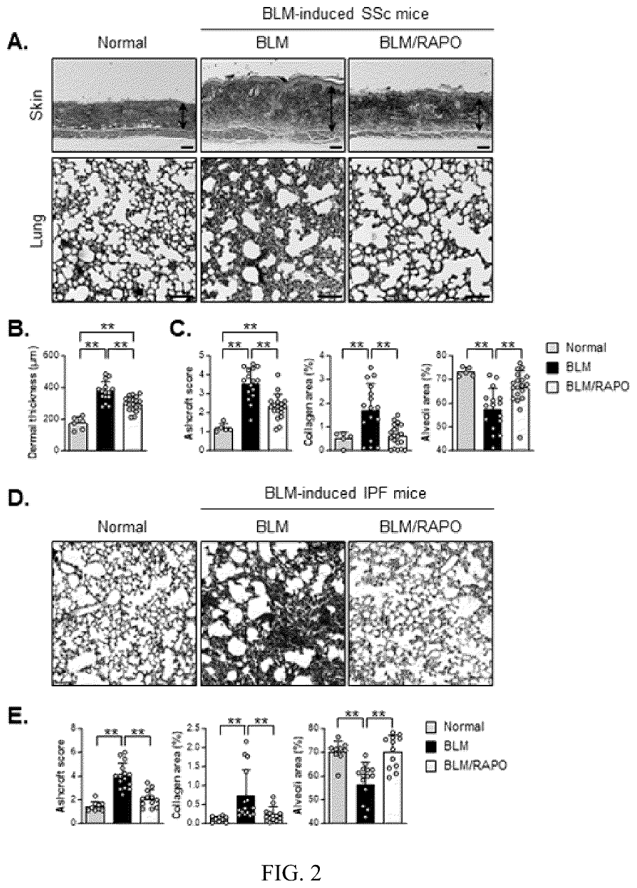

The result showed that skin and lung fibrosis were reproducibly suppressed in systemic sclerosis mice (BLM/RAPO group) administered Bifidobacterium longum RAPO, compared to systemic sclerosis mice (BLM group) not administered the strain, like the result of FIG. 1 (A to C of FIG. 2). In addition, the result showed that lung fibrosis was suppressed in idiopathic pulmonary fibrosis mice administered with Bifidobacterium longum RAPO, compared to idiopathic pulmonary fibrosis mice (BLM/RAPO group) not administered the strain (BLM group) (D and E of FIG. 2), which indicates that Bifidobacterium longum RAPO is effective in alleviating lung fibrosis in both mouse disease models.

3) Analysis of Single/Combined Efficacy of Conventional Drugs for Pulmonary Fibrosis and Bifidobacterium longum RAPO (KCTC13773BP) in Idiopathic Pulmonary Fibrosis Test Model

To determine whether or not administration of a combination of pirfenidone (PFD), which is a conventional approved and used drug for pulmonary fibrosis, with Bifidobacterium longum RAPO, has a synergistic therapeutic effect due to Bifidobacterium longum RAPO, the inhibitory effects on pulmonary fibrosis after single and combination administration of PFD and Bifidobacterium longum were compared and evaluated in mice with idiopathic pulmonary fibrosis.

Idiopathic pulmonary fibrosis was induced by a single intratracheal injection of 50 μL (2 U/mouse kg) of BLM into 8-week-old male C57BL/6 mice to induce pulmonary fibrosis. Pirfenidone (PFD) and Bifidobacterium longum RAPO were administered orally 5 times a week for a total of 2 weeks, starting 7 days after inducing pulmonary fibrosis with BLM until the 21st day, the end date of the experiment. The administration groups and doses were as follows: 1) Normal control group: normal mice that were not administered any treatment; 2) disease control group: IPF mice administered vehicle containing neither PFD nor Bifidobacterium longum RAPO; 3) P100 group: IPF mice administered PFD 100 mg/mouse kg; 4) P200 group: IPF mice administered PFD 200 mg/mouse kg; 5) RAPO-A group: IPF mice administered Bifidobacterium longum RAPO 1×10⁸ CFU/mouse; 6) RAPO-B group: IPF mice administered Bifidobacterium longum RAPO 5×10⁸ CFU/mouse; 8) Comb A group: IPF mice administered a combination of PFD 100 mg/mouse kg and Bifidobacterium longum RAPO 1×10⁸ CFU/mouse; 7) Comb B group: IPF mice administered a combination of 100 mg/mouse kg and Bifidobacterium longum RAPO 5×10⁸ CFU/mouse. Efficacy evaluation was performed as follows. Lung tissues of mice with fibrosis induced on the 21st day after the start of BLM administration were sampled and fixed with formalin to form paraffin tissue blocks. The formed paraffin blocks were cut into 5 μm thick sections with a microtome and stained with Masson-Trichrome (MT), and the degree of pulmonary fibrosis was evaluated by analysis of an Ashcroft score, a collagen area, and an alveoli area.

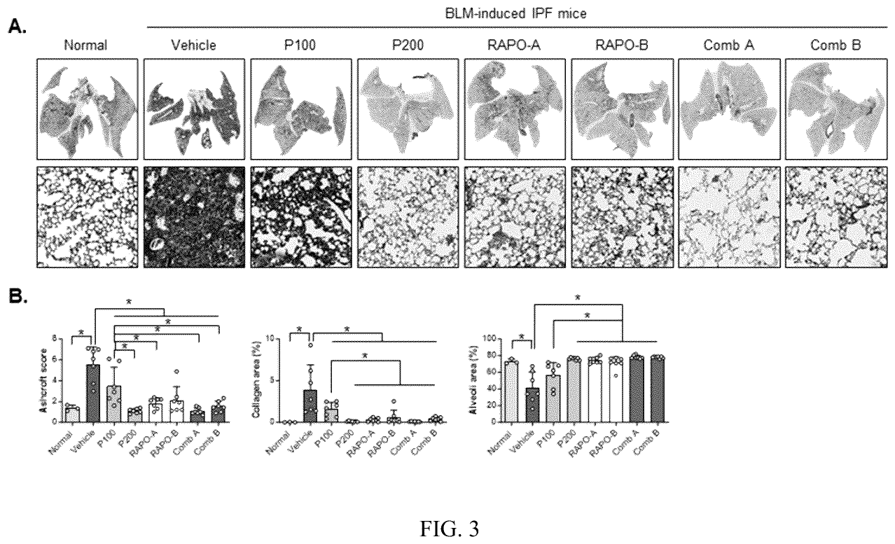

As a result, as shown in FIG. 3, the idiopathic pulmonary fibrosis mouse group (P200 group) that was administered PFD 200 mg/mouse kg alone, which is the level used in actual patients, significantly alleviated pulmonary fibrosis, compared to the idiopathic pulmonary fibrosis mouse group (vehicle group) that was not administered PFD or Bifidobacterium longum RAPO. The idiopathic pulmonary fibrosis mouse group (P100 group) administered PFD 100 mg/mouse kg alone, which is half the level, also alleviated pulmonary fibrosis, as can be seen from improvement in some pulmonary fibrosis parameters such as an Ashcroft score and a collagen area, compared to the vehicle group, but did not ameliorate pulmonary fibrosis comparable to the P200 group.

Meanwhile, the idiopathic pulmonary fibrosis mouse groups (RAPO-A and -B groups) administered Bifidobacterium longum RAPO 1×10⁸ CFU/mouse and 5×10⁸ CFU/mouse alone also ameliorated pulmonary fibrosis and the effect of ameliorating pulmonary fibrosis was significantly increased compared to the P100 group.

In order to determine whether or not reducing the dosage of PFD in patients who have side effects or are incurable to PFD can reduce side effects while exhibiting similar or improved therapeutic effects to the conventional PFD, an experiment with a combination of the P100 group and Bifidobacterium longum RAPO-A and -B groups was conducted.

The result shows that both the Comb A group and the Comb B group had an effect of ameliorating pulmonary fibrosis compared to the vehicle group and the P100 group, and the effects thereof are improved compared to the RAPO-A and RAPO-B groups that were administered Bifidobacterium longum RAPO alone, although not significant, and the Comb A group and the Comb B group had a similar degree of amelioration to the P200 group, which corresponds to a conventional PFD dosage, which supports that the combined therapy of PFD and Bifidobacterium longum RAPO is very useful in the treatment of idiopathic pulmonary fibrosis.

### Experimental Example 1: Analysis of Expression of Fibrosis Marker Protein of Bifidobacterium longum RAPO (KCTC13773BP) in Animal Test Model (Mouse)

The expression of α-smooth muscle actin antibody (α-SMA), which is a marker of differentiation into myofibroblasts in the skin and lung tissues of normal mice, systemic sclerosis mice, and systemic sclerosis mice administered with Bifidobacterium longum RAPO, was measured using immunofluorescence or immunohistochemical staining methods.

For immunofluorescence staining, deparaffinized skin and lung tissue slides were added to antigen retrieval buffer (10 mM sodium citrate+0.05% Tween-20, pH 6.0) at 95° C. and were allowed to stand at room temperature for 40 minutes to expose the antigen. The skin and lung tissue slides were each washed three times with PBS for 10 minutes and were then blocked with PBS containing 1.5% normal horse serum at room temperature for 1 hour. Then, the skin and lung tissue slides were reacted with an α-SMA primary antibody (1 μg/mL, ab7817, Abcam) at 4° C. for 16 hours and then reacted with a secondary fluorescent antibody anti-mouse Alexa Fluor 488 (1:500, A-21202, Thermo Fisher Scientific) at room temperature for 2 hours. The result was mounted with a mounting solution containing DAPI (P36935, Thermo Fisher Scientific), the expression of α-SMA was observed under a fluorescence microscope (Nikon Eclipse Ni-U microscope; Nikon Instruments), and the fluorescence intensity was analyzed using ImageJ software.

Immunohistochemical staining was performed in the same manner as described with reference to immunofluorescence staining. The antigen of the slide tissue was exposed, and the sections were treated with a 100% methanol solution containing 0.3% hydrogen peroxide for 30 minutes, and then with PBS containing 1.5% normal horse serum at room temperature for 1 hour. Then, the sections were reacted with a primary antibody α-SMA (0.034 μg/mL, ab7817, Abcam) at 4° C. for 16 hours, and with a biotinylated secondary antibody (BA-9200, Vector Laboratories) at room temperature for 90 minutes. The slides were treated with VECTASTAIN Elite ABC reagent at room temperature for 1 hour to induce avidin-biotin binding, were stained with a 0.05% diaminobenzidine (D5637, Sigma-Aldrich) solution and then counterstained with hematoxylin. Then, the expression of α-SMA was observed using an optical microscope (Nikon) and the color intensity was analyzed using ImageJ software.

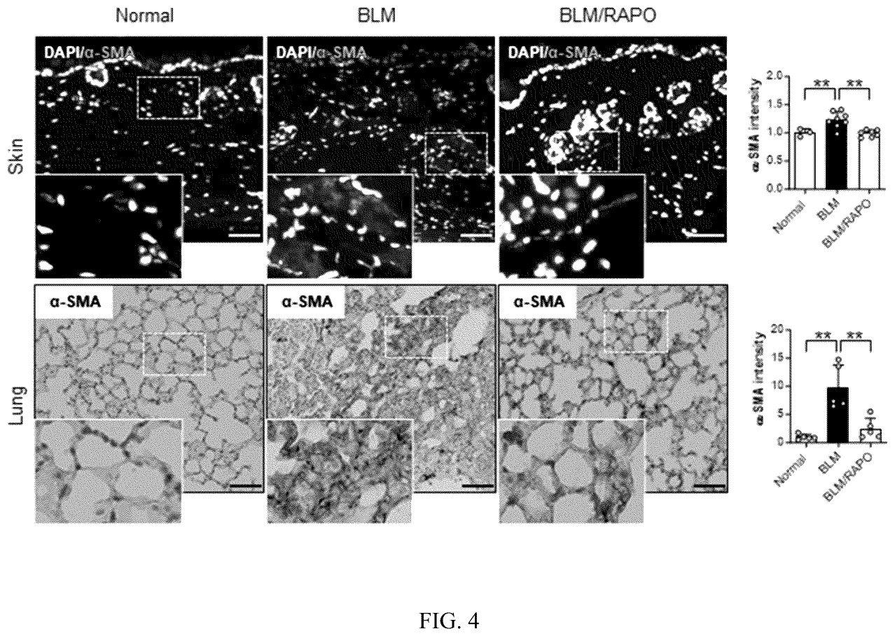

As a result, as can be seen from FIG. 4, systemic sclerosis mice (BLM group) that were not treated with Bifidobacterium longum RAPO increased the expression of α-SMA in both skin and lung tissues, compared to the normal mice (Normal group). On the other hand, systemic sclerosis mice (BLM/RAPO group) that were treated with Bifidobacterium longum RAPO effectively reduced the expression of α-SMA in the skin and lung tissues, inhibited differentiation into myofibroblasts in the skin and lung tissues, and were thus involved in ameliorating fibrosis.

### Experimental Example 2: Macrophage Analysis, Inflammatory Substance Concentration Measurement, Fibrosis/Inflammation Marker Gene Expression, and Inflammatory Monocyte Analysis of Bifidobacterium longum RAPO (KCTC13773BP) in Animal Test Model (Mouse)

1) Macrophage Analysis of Bifidobacterium longum RAPO (KCTC13773BP)

Macrophage infiltration and changes in skin tissue and bronchoalveolar lavage (BAL) of normal mice, systemic sclerosis mice, and systemic sclerosis mice administered Bifidobacterium longum RAPO were evaluated. The details of the method is as follows.

To evaluate macrophages in skin tissue, skin tissue section slides were immunofluorescently stained with primary antibodies against CD11b (1:200, sc-23937, Santa Cruz Biotechnology) and CX3CR1 (1:200, sc-377227, Santa Cruz Biotechnology), and observed under a fluorescence microscope, and the fluorescence intensity of CD11b⁺CX3CR1⁺ macrophages was analyzed using ImageJ software. Bronchoalveolar lavage fluid was collected by inserting a 20-gauge venous catheter into the mouse trachea, injecting 1 mL of PBS containing 0.1 mM EDTA into the catheter, and washing the catheter twice. The total volume of the collected lavage fluid was measured, and the supernatant was centrifuged at 400 g for 7 minutes to separately obtain the supernatant and the cells. The total cell number was measured using a TC20 automatic cell counter (Bio-Rad) and leukocytes were subjected to cytospin and Diff-Quik staining and then differentially counted.

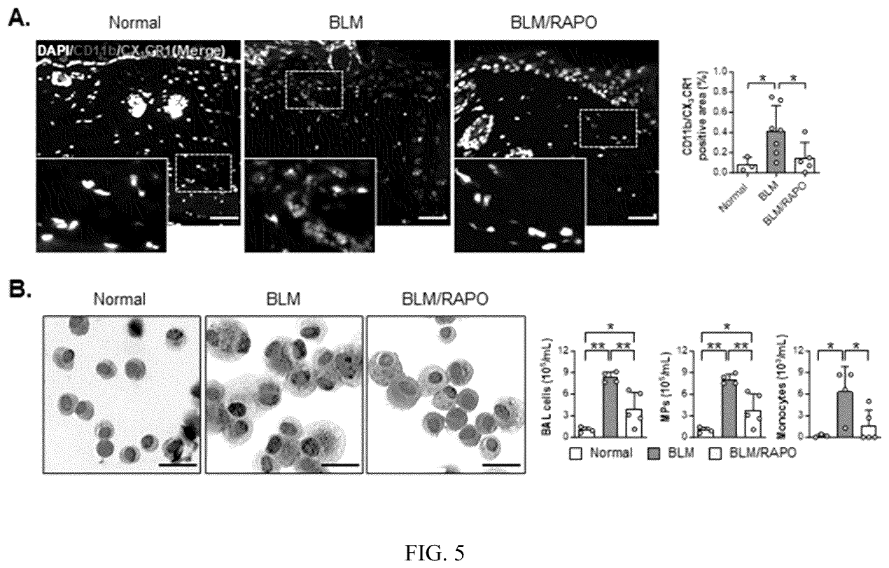

The degree of macrophage infiltration was compared through fluorescent staining of CD11b and CX3CR1 macrophage markers in skin tissue. It can be seen from A of FIG. 5 that the BLM group had an increase in CD11b⁺CX3CR1⁺ macrophages in skin tissue compared to normal mice and the increased CD11b⁺CX3CR1⁺ macrophages in the skin of the BLM group decreased after administration of Bifidobacterium longum RAPO.

In addition, the morphological changes of cells present in the bronchoalveolar fluid were evaluated. As can be seen from B of FIG. 5, the main cells constituting the bronchoalveolar fluid cells of the BLM group were macrophages, most macrophages were enlarged in size and had activated changes with vacuolated cytoplasm, and these changes were alleviated by administration of Bifidobacterium longum RAPO. In addition, the total cell count, macrophage count, and monocyte count in the bronchoalveolar fluid of the BLM group were significantly increased compared to normal mice, and this increase was effectively reduced by administration of Bifidobacterium longum RAPO.

2) Measurement of Concentration of Inflammatory Substances in Bifidobacterium longum RAPO (KCTC13773BP)

The bronchoalveolar lavage fluid was collected from normal mice, systemic sclerosis mice, and systemic sclerosis mice administered Bifidobacterium longum RAPO, and the level of inflammatory substances contained in the supernatant excluding cells was measured. The concentrations of total protein (Bradford assay, Bio-rad) and interleukin-6 (IL-6, DuoSet ELISA kits, R&D System) were measured in accordance with the protocol provided by the manufacturer.

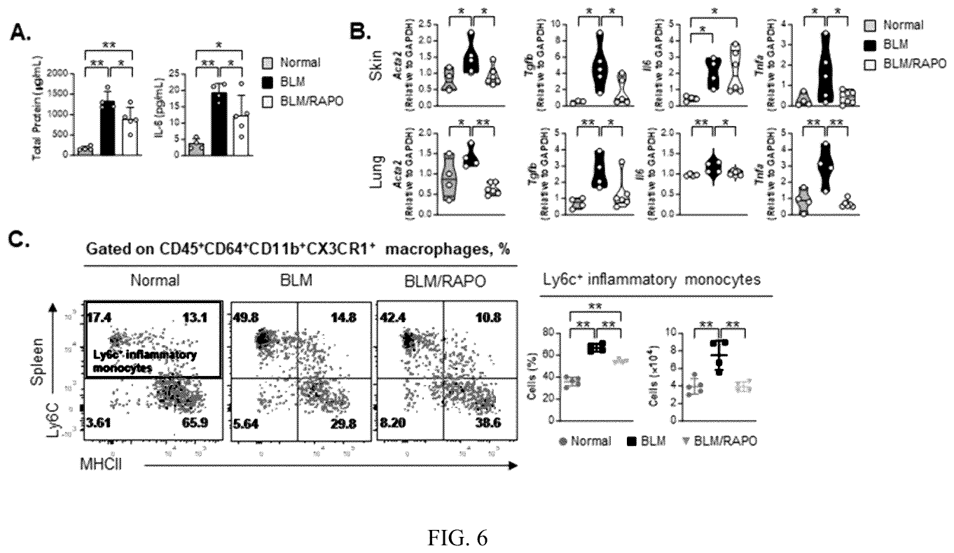

The level of inflammatory substances in the bronchoalveolar supernatant was measured. The result showed that the BLM group exhibited considerable increases in total protein and IL-6 compared to normal mice, as shown in A of FIG. 6. These inflammatory substances increased in the BLM group were effectively reduced by administration of Bifidobacterium longum RAPO.

3) Analysis of Fibrosis/Inflammation Marker Gene Expression of Bifidobacterium longum RAPO (KCTC13773BP)

The expression of fibrosis/inflammation cytokine genes was evaluated in the skin and lung tissues of normal mice, systemic sclerosis mice, and systemic sclerosis mice administered Bifidobacterium longum RAPO. Total RNA was extracted from the skin and lung tissues of the mice using TRIzol reagent (Thermo Fisher Scientific) and cDNA was synthesized using 1 μg of total RNA using iScript cDNA synthesis kit (1708891, Bio-Rad).

Target gene amplification was performed by qPCR with a ViiA 7 real-time PCR detection system (Applied Biosystems) using Taqman (4369016, Thermo Fisher Scientific) or SYBR Green PCR master mix (4367659, Thermo Fisher Scientific). The expression level of target genes was standardized using glyceraldehyde 3-phosphate dehydrogenase (Gapdh) as an internal control, and the relative expression level was analyzed using a 2^{-ΔCt} comparative method. The target gene primers for Taqman (Thermo Fisher Scientific) used in qPCR were mActa2 (Mm00725412_s1), mTgfb1 (Mm01178820_ml), and mGapdh (Mm99999915_g1), and the primers for SYBR Green are as shown in Table 1 below.

TABLE 1

| | | | | Sequence |
|---|---|---|---|---|
| Species | Gene symbol | Direction | Sequence | information |
| Mouse | I16 | Forward | 5′- GGCCTTCCCTACTTCACAAG - | SEQ ID NO: 1 |
| | | | 3′ | |
| | | Reverse | 5′- ATTTCCACGATTTCCCAGAG -3′ | SEQ ID NO: 2 |
| Mouse | Il1b | Forward | 5′- CTGGTGTGTGACGTTCCCATTA - | SEQ ID NO: 3 |
| | | | 3′ | |
| | | Reverse | 5′- CCGACAGCACGAGGCTTT -3′ | SEQ ID NO: 4 |
| Mouse | Tnfa | Forward | 5′- | SEQ ID NO: 5 |
| | | | GACCCTCACACTCAGATCATCT - | |
| | | | 3′ | |
| | | Reverse | 5′- CCTCCACTTGGTGGTTTGCT -3′ | SEQ ID NO: 6 |
| Mouse | Ccl2 | Forward | 5′- CCAGCCTACTCATTGGGAT -3′ | SEQ ID NO: 7 |
| | | Reverse | 5′- GGGCCTGCTGTTCACAGTT -3′ | SEQ ID NO: 8 |
| Mouse | Gapdh | Forward | 5′- ACCCAGAAGACTGTGGATGG - | SEQ ID NO: 9 |
| | | | 3′ | |
| | | Reverse | 5′- ACACATTGGGGGTAGGAACA - | SEQ ID NO: 10 |
| | | | 3′ | |

As a result, as shown in B of FIG. 6, Acta2 and Tgfb as fibrosis genes, and Il6 and Tnfa as inflammatory cytokine genes were overexpressed in both skin and lung tissue of the BLM group compared to normal mice, and most of the fibrosis/inflammatory genes overexpressed in the skin and lung tissue of the BLM group were significantly suppressed by administration of Bifidobacterium longum RAPO.

4) Analysis of Inflammatory Mononuclear Cells of Bifidobacterium longum RAPO (KCTC13773BP)

The spleens of normal mice, systemic sclerosis mice, and systemic sclerosis mice administered Bifidobacterium longum RAPO were removed, pulverized, and then centrifuged at 1,500 rpm for 5 minutes to separate spleen cells. These cells were reacted with ACK lysis buffer for 5 minutes to lyse red blood cells, and then centrifuged at 1,500 rpm for 5 minutes to separate cells from which red blood cells had been removed. The separated cells were stained with fluorescently conjugated antibodies (Table 2) at 4° C. for 30 minutes and analyzed using an LSRFortessa™ X-20 flow cytometer (BD Biosciences). FlowJo software (Tree Star Inc.) was used for additional data analysis.

TABLE 2

| Antibodies | Source |
|---|---|
| FVS510 | BD Bioscience |
| Anti-mouse CD45, APC/Cy7, clone 30-F11 | BD Bioscience |
| Anti-mouse CD64, BV780, clone X54-5/7.1 | BD Bioscience |
| Anti-mouse CD11b, BB515, clone M1/70 | BD Bioscience |
| Anti-mouse CD11c, BB700, clone HL3 | BD Bioscience |
| Anti-mouse CX3CR1, APC, clone SA011F11 | BioLegend |
| Anti-mouse MHCII (I-A/I-E), BV421, clone | BioLegend |
| M5/114.15.2 | |
| Anti-mouse Ly-6C, BV605, clone AL-21 | BD Bioscience |

The proportion (%) and number of CD45⁺CD64⁺CD11b⁺CX3CR1⁺Ly6C⁺ inflammatory monocytes in the mouse spleen were analyzed using a flow cytometer. As shown in C of FIG. 6, the proportion of Ly6C⁺ monocytes in CD45⁺CD64⁺CD11b⁺CX3CR1⁺ macrophages and the number of inflammatory monocytes were increased in the spleen of the BLM group compared to normal mice, and the increase was reduced by administration of Bifidobacterium longum RAPO.

### Experimental Example 3: Analysis of Effect of Bifidobacterium longum RAPO (KCTC13773BP) on Fecal Microbiome in Animal Test Model (Mouse)

To determine whether or not Bifidobacterium longum RAPO affected the composition of intestinal microorganisms in systemic sclerosis mice, 16S rRNA sequencing was performed on fecal samples from normal mice, systemic sclerosis mice, and systemic sclerosis mice administered B. longum RAPO, and total bacterial genomic DNA was extracted using a MagMAX® microbiome ultra nucleic acid isolation kit of a KingFisher Flex system (Thermo Fisher Scientific). The library was constructed in accordance with a 16S metagenomic sequencing library preparation guide protocol (Illumina, San Diego, CA, USA). Variable regions 3 and 4 of the bacterial 16S rRNA gene were amplified using primers targeting MiSeq 341F and 805R. Then, the amplicons were sequenced using an Illumina MiSeq System (2×300 bp paired-end reads) and raw sequence reads were analyzed using QIIME2 software. The microbial diversity and population distribution, and the relative distribution rate of each microorganism were analyzed using a QIIME2 pipeline and R software.

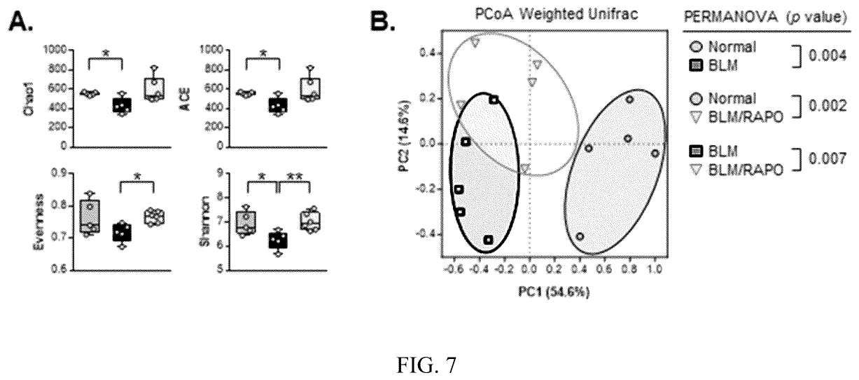

The diversity of the microbial population through four alpha diversity indices was evaluated. As shown in A of FIG. 7, the result shows that the alpha diversity indices of the BLM group decrease compared to the normal mice, whereas the BLM/RAPO group recovered the alpha diversity indices decreased in the BLM group. In particular, it was found that the alpha diversity evaluated by the Shannon index was significantly improved by Bifidobacterium longum RAPO.

In addition, as shown in B of FIG. 7, the beta diversity analysis using PCoA analysis shows that there was a significant difference in the overall fecal microbial population composition between the test groups. Analysis of the diversity of these microbial populations showed that Bifidobacterium longum RAPO affects the gut microbiome of systemic sclerosis mice.

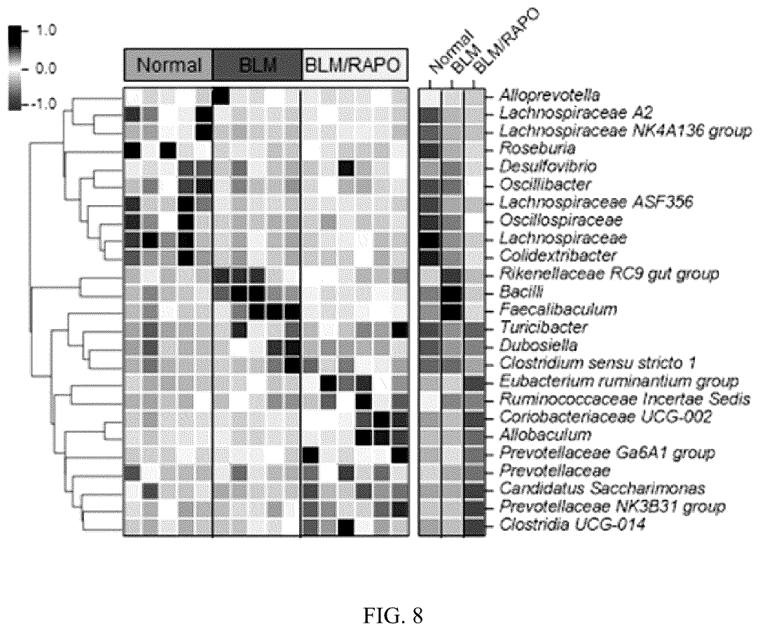

More specifically, the relative distribution of microorganisms that significantly increased or decreased between the experimental groups at the genus level was analyzed. As shown in FIG. 8, the result shows that most of the microorganisms with significant differences are associated with the production of short chain fatty acids (SCFAs). In particular, SCFA-producing microorganisms were abundant in the normal group and the BLM/RAPO group, which means that administration of Bifidobacterium longum RAPO may increase the SCFA-producing microorganisms in the intestines of systemic sclerosis mice and thus affect the alleviation of skin and pulmonary fibrosis.

### Experimental Example 4: In Vitro Anti-Inflammatory/Antifibrotic Efficacy Analysis of Bifidobacterium longum RAPO (KCTC13773BP) and Metabolites Thereof in Macrophages and Fibroblasts

The in vitro anti-inflammatory/antifibrotic efficacy of Bifidobacterium longum RAPO (KCTC13773BP) and metabolites thereof in macrophages and fibroblasts was analyzed.

### 1) Culture and Treatment of Cell Line

Mouse macrophage cell line RAW 264.7 was suspended in Dulbecco's modified eagle's medium (DMEM, GIBCO) containing 10% fetal bovine serum (FBS), 100 U/mL penicillin, and 100 mg/mL streptomycin (GIBCO), seeded into 24-well plates at 1×10⁵ cells per well, and cultured at 37° C. and 5% CO₂ for 24 hours. Then, medium (0, 0.01, or 0.1%) cultured with various amounts of heat-killed (HK)-Bifidobacterium longum RAPO (multiplicity of infection, MOI 0, 0.1, or 1) or Bifidobacterium longum RAPO was added to the plates containing the cells and cocultured for 20 hours. Then, lipopolysaccharide (LPS, clone O55: B5, Sigma-Aldrich) was added to each plate at a concentration of 1 μg/mL for cell stimulation and then further cultured for 4 hours.

Primary normal human dermal fibroblasts (NHDF, PCS-201-012, ATCC) were suspended in DMEM (+10% FBS, 100 U/mL penicillin, 100 mg/mL streptomycin), and then seeded into 6-well plates at 2×10⁵ cells per well, and cultured at 37° C. and 5% CO₂ for 24 hours. Then, the medium (0 or 2%) cultured with Bifidobacterium longum RAPO was added to the plate containing the cells, and at the same time, human recombinant transforming growth factor-beta1 (hTGF-βR&D System) was added at a level of 10 ng/mL and cultured for 48 or 72 hours.

Normal lung fibroblasts (normal-LFs, LL24, CCL151, ATCC) and lung fibroblasts (IPF-LFs, LL29, CCL134, ATCC) from patients diagnosed with IPF were suspended in Ham's F-12 medium (+10% FBS, 100 U/mL penicillin, 100 mg/mL streptomycin), seeded on 6-well plates at 2×10⁵ cells per well and cultured at 37° C. and 5% CO₂ for 24 hours. Then, the medium (0 or 2%) cultured with Bifidobacterium longum RAPO was added to the plate containing the cells and at the same time, human recombinant transforming growth factor-beta1 (hTGF-βR&D System) was added in a level of 5 ng/mL and cultured for 72 hours.

2) Evaluation of Anti-Inflammatory Efficacy of Bifidobacterium longum RAPO (KCTC13773BP) and Metabolites Thereof in Macrophages

Whether or not Bifidobacterium longum RAPO or secretions/metabolites thereof could directly participate in regulating macrophage activation in inflammatory conditions was determined in vivo. For this purpose, total RNA was extracted from RAW 264.7 treated under various conditions with LPS, HK-RAPO, and RAPO culture media using TRIzol reagent (Thermo Fisher Scientific) and cDNA was synthesized using iScript cDNA synthesis kit (1708891, Bio-Rad) with 0.5 μg of total RNA. Target gene amplification was performed by qPCR using SYBR Green PCR master mix with ViiA 7 real-time PCR detection system. Target gene expression levels were normalized using Gapdh as an internal control and the relative expression levels were analyzed using the 2^{-ΔΔCt} comparative method. The primers for SYBR Green used in qPCR are shown in Table 1 (see the table above).

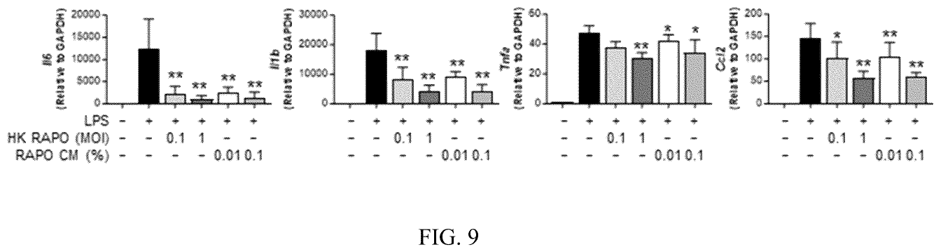

As shown in FIG. 9, the result shows that the expression of Il6, Il1b, Tnfa, and Ccl2 genes increased by LPS was suppressed when RAW 264.7 cells were treated with HK-Bifidobacterium longum RAPO and Bifidobacterium longum RAPO culture solution (RAPO CM). This supports that Bifidobacterium longum RAPO or secretion/metabolites thereof have an inhibitory efficacy against macrophage activation and the inhibition of macrophage activation may affect prevention of progression into fibrosis.

3) Evaluation of Antifibrotic Efficacy of Bifidobacterium longum RAPO (KCTC13773BP) and Metabolites Thereof in Fibroblasts

Whether or not the secretion/metabolites of Bifidobacterium longum RAPO could be involved in the fibrotic process was determined in vitro. For this purpose, NHDF, LL24, and LL29 cells treated under the conditions of hTGF-β and RAPO culture medium were lysed by treatment with RIPA buffer (89900, Thermo Fisher Scientific) containing a protease/phosphatase inhibitor cocktail (GenDEPOT) at 4° C. for 2 hours to extract proteins. After protein assay using bicinchoninic acid (BCA) protein assay (Pierce), 20 μg of the protein was loaded onto 12% SDS-PAGE, electrophoresed, and transferred to polyvinylidene fluoride membranes (PVDF, IPVH00010, Merck Millipore). The PVDF membrane was blocked in TBS buffer containing 5% skimmed milk and 0.1% Tween for 1 hour and then reacted with a primary antibody against α-SMA (0.341 μg/mL, ab7817, Abcam) at 4° C. for 16 hours. Then, the membrane was treated with a horseradish peroxidase-conjugated secondary antibody (1:3000, Bio-Rad) at room temperature for 1 hour and protein expression was identified using ECL western blotting substrates (Bio-Rad). β-actin was used as an internal control.

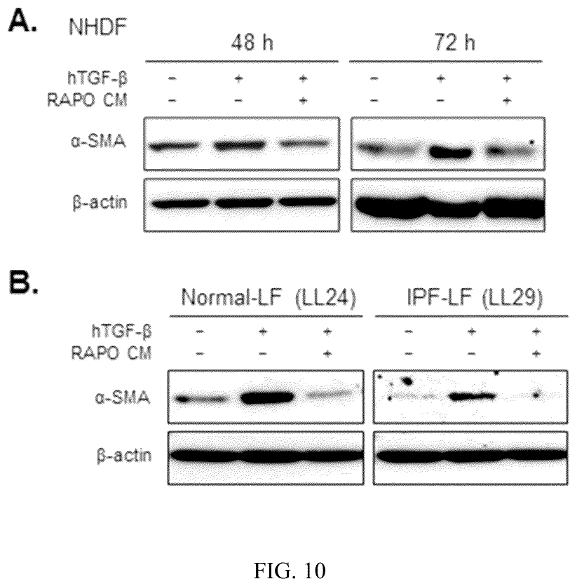

As shown in FIG. 10, the result showed that the overexpression of α-SMA protein induced by hTGF-β1 was suppressed when NHDF, and LL24 and LL29 cells, which are skin and lung fibroblast cells, were treated with RAPO culture medium (RAPO CM). In other words, the inhibitory efficacy of secretions/metabolites from B. longum RAPO on differentiation into myofibroblasts was verified and it appears that secretions and metabolites of B. longum RAPO contribute to suppression of fibrosis.

As apparent from the fore-going, the present invention provides a composition containing Bifidobacterium longum RAPO (KCTC13773BP) as an active ingredient that is capable of controlling inflammatory monocyte and macrophage infiltration and inflammatory response, of facilitating an increase in short-chain fatty acid producing microorganisms, and of exhibiting an effect of ameliorating, preventing or treating systemic sclerosis and pulmonary fibrosis.

What is claimed is:

1. A method for preventing, ameliorating or treating systemic sclerosis, the method comprising administering to a subject in need thereof a composition comprising Bifidobacterium longum RAPO (KCTC13773BP) as an active ingredient.

2. The method according to claim 1, wherein the subject requires preventing, ameliorating or treating systemic sclerosis and pulmonary fibrosis.
